(19) United States
(12) Patent Application Publication
(10) Pub. No.: US 2009/0247595 A1
(43) Pub. Date: Oct. 1, 2009

(54) Process for the preparation of tetrazolyl compounds

(76) Inventor: Nuria Soldevilla Madrid, Barcelona (ES)

Correspondence Address: FROMMER LAWRENCE & HAUG, 745 FIFTH AVENUE- 10TH FL., NEW YORK, NY, 10151, US

(21) Appl. No.: 11/921,677

(22) PCT Filed: Jun. 6, 2006
(86) PCT No.: PCT/IB2006/003936

§ 371 Date: Jun. 1, 2009

Related U.S. Application Data
(60) Provisional application No. 60/687,305, filed on Jun. 6, 2005.
(60) Provisional application No. 60/771,466, filed on Feb. 9, 2006.

(51) Int. Cl. A61K 31/4184; C07D 403/10

(52) U.S. Cl. 514/381; 548/253

(57) ABSTRACT

The invention provides a method for preparing candesartan cilexetil and related tetrazolyl compounds. More particularly, the invention relates to the preparation of candesartan cilexetil and related tetrazolyl compounds and includes a method of removing a protective group (e.g., triphenylmethane (trityl) protecting group) from an N-protected tetrazolyl compound using a Lewis acid in an inert solvent and in the presence of an alcohol (e.g., reacting an N-protected tetrazolyl compound with ZnCl₂ in the presence of an alcohol).

### CROSS REFERENCE TO RELATED APPLICATIONS

This application claims priority to U.S. Provisional Application Nos. 60/687,305, filed Jun. 6, 2005 and 60/771,466, filed Feb. 9, 2006, which applications are expressly incorporated herein by reference in their entirety.

### BACKGROUND OF THE INVENTION

1. Field of the Invention

The invention relates, in general, to the preparation of candesartan cilexetil and related tetrazolyl compounds. More particularly, the invention relates to the preparation of candesartan cilexetil and related tetrazolyl compounds and includes a method of removing a protective group (e.g., triphenylmethane (trityl) protecting group) from an N-protected tetrazolyl compound using a Lewis acid in an inert solvent and in the presence of an alcohol (e.g., reacting an N-protected tetrazolyl compound with ZnCl₂ in the presence of an alcohol).

2. Relevant Background

The chemical name for candesartan cilexetil is 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate.

Candesartan cilexetil can be produced as described in U.S. Pat. Nos. 5,196,444 (“the '444 patent”) and 5,763,619 (“the '619 patent”). In each of these patents, candesartan cilexetil is prepared by deprotecting N-trityl candesartan cilexetil (i.e., removing the N-trityl protecting group). The '444 patent describes a method of using hydrochloric acid in methanol to remove the trityl protecting group. This method however, results in low yields, and the resulting product has to be chromatographically purified. The '444 patent further describes a method for preparing the intermediate N-trityl candesartan cilexetil (i.e., candesartan cilexetil trityl) involving the reaction of 2-ethoxy-1-[[2′-(1-triphenylmethyl-1H-tetrazol-5-yl)biphenyl-4-yl]methyl]-1H-benzimidazole-7-carboxylic (i.e., candesartan trityl) with cyclohexyl-1-iodoethyl carbonate in the presence of potassium carbonate and N,N-dimethylformamide.

The '619 patent improved upon the process described in the '444 patent by utilizing anhydrous hydrogen chloride in methanol to remove the trityl group such that the proportion of the decomposition products is lower and the yield higher. According to the '619 patent, an advantage of this method for removing the trityl group is that it minimizes and/or eliminates the unintended removal of other hydrolysable functional groups (e.g., esterified carboxyl groups, alkoxy groups). This method, however, exhibits yield fluctuations, and the use of anhydrous hydrogen chloride can be corrosiveness to the reaction medium.

U.S. Pat. No. 6,608,210 describes a method of removing a trityl protecting group with hydrochloric acid in aqueous acetic acid.

WO 2005/021535 describes a method for removing the trityl protecting group by solvolysis in a neutral or slightly basic anhydrous alcohol medium. This method, however, requires on the order of several hours of reaction at reflux temperature (e.g., 10 hours for preparing irbesartan and valsartan and 24 hours for preparing candesartan cilexetil).

WO 2005/037821 A2 describes a method of removing the trityl protecting group with an organic acid in methanol without the presence of an acid.

WO 05/051928A1 describes a method of removing the trityl protecting group with organic acids in anhydrous conditions.

J. Med. Chem., 36, 2343 (1993) and U.S. Pat. No. 6,177,587 describe a method for preparing candesartan cilexetil trityl involving the reaction of candesartan trityl with chloroethyl cyclohexyl carbonate in the presence of potassium carbonate, potassium iodide and N,N-dimethylformamide.

WO 2005/037821 A2 describes a method for preparing candesartan cilexetil trityl involving the reaction of candesartan trityl with chloroethyl cyclohexyl carbonate in a low boiling organic solvent (e.g. toluene) and in the presence of a phase transfer catalyst (e.g. tetrabutylammoniumhydrogensulfate).

Candesartan cilexetil is poorly soluble in water, which necessitates special formulation procedures for achieving a desired pharmacokinetic profile. In general, low solubility compounds can be problematic in the pharmaceuticals arts from a formulations perspective. In this regard, specific surface area can affect the solubility properties of a compound, like candesartan cilexetil. The surface area of a solid material provides information about the void spaces on the surfaces of individual particles or aggregates of particles. Factors such as chemical activity, adsorption, dissolution, and bioavailabilty of the drug may depend on the surface of the solid. In view of the foregoing, there is a need in the medical arts for candesartan cilexetil with a specific surface area.

### SUMMARY OF THE INVENTION

The invention provides a method for preparing candesartan cilexetil and related tetrazolyl compounds. More particularly, the invention relates to the preparation of candesartan cilexetil and related tetrazolyl compounds and includes a method of removing a protective group (e.g., triphenylmethane (trityl) protecting group) from an N-protected tetrazolyl compound using a Lewis acid in an inert solvent and in the presence of an alcohol (e.g., reacting an N-protected tetrazolyl compound with ZnCl₂ in the presence of an alcohol).

One aspect of the invention provides a process for removing a protective group (e.g., triphenylmethane (trityl) protecting group) of an N-protected tetrazolyl compound that includes reacting an N-protected tetrazolyl compound with a Lewis acid in the presence of an alcohol.

In another aspect of the invention, there is provided a process for removing a protective group (e.g., triphenylmethane (trityl) protecting group) of an N-protected tetrazolyl compound that includes reacting an N-protected tetrazolyl compound with ZnCl₂ in the presence of an alcohol.

In another aspect of the invention, there is provided a process for producing a tetrazolyl compound that includes reacting an N-protected tetrazolyl compound with a Lewis acid in the presence of an alcohol.

In another aspect of the invention, there is provided a process for producing a tetrazolyl compound that includes reacting an N-protected tetrazolyl compound with ZnCl₂ in the presence of an alcohol.

In another aspect of the invention, there is provided an improved process for the production of N-protected tetrazolyl compounds using a phase transfer catalyst. In particular, the invention includes preparing the intermediate 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl) by condensing 2-ethoxy-1-[[2′-(1-triphenylmethyl-1H-tetrazol-5-yl)biphenyl-4-yl]methyl]-1H-benzimidazole-7-carboxylic acid with chloroethyl cyclohexyl carbonate in refluxing tetrahydrofuran and in the presence of benzyltriethylammonium chloride and potassium carbonate. The obtained candesartan cilexetil trityl can be recrystallized and isolated from an alcohol, preferably isopropyl alcohol.

In another aspect of the invention, there is provided an improved process for producing the N-protected tetrazolyl compounds using an organic solvent of high boiling point. In particular, the invention includes preparing the intermediate 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl) by condensing 2-ethoxy-1-[[2′-(1-triphenylmethyl-1H-tetrazol-5-yl)biphenyl-4-yl]methyl]-1H-benzimidazole-7-carboxylic acid with chloroethyl cyclohexyl carbonate in an organic solvent of high boiling point in the presence of potassium carbonate. The preferred organic solvents of high boiling point are N-methyl-2-pyrrolidinone (NMP), dimethyl sulfoxide (DMSO) and mixtures thereof. The obtained candesartan cilexetil trityl can be recrystallized and isolated from an organic acetate solvent, preferably isopropyl acetate.

In another aspect of the invention, there is provided a powder composition of candesartan cilexetil having a specific surface area of approximately 1 to approximately 3 m²/g.

In another aspect of the invention, there is provided candesartan cilexetil which is substantially free of solvent, for example containing less than approximately 2%, preferably less than approximately 1%, more preferably less than approximately 0.5%, most preferably less than approximately 0.1% of solvent.

Compounds produced according to one or more aspects of the invention can be used as therapeutics for treating hypertension and circulatory diseases (e.g., heart failure, strokes, cerebral apoplexy, nephropathy and nephritis).

The invention is advantageously useful for preparing tetrazolyl compounds having at least one hydrolysable group in addition to the protective group on the N-protected tetrazolyl compound.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention provides a method for preparing candesartan cilexetil and related tetrazolyl compounds. More particularly, the invention relates to the preparation of candesartan cilexetil and related tetrazolyl compounds and includes a method for removing a protective group (e.g., triphenylmethane (trityl) protecting group) from an N-protected tetrazolyl compound by solvolysis using a Lewis acid in an inert solvent and in the presence of an alcohol (e.g., reacting an N-protected tetrazolyl compound with ZnCl₂ in the presence of an alcohol).

The invention includes subjecting an N-protected tetrazolyl compound to solvolysis by suspending the N-protected tetrazolyl compound in an inert solvent (e.g. toluene, tetrahydrofuran, acetone, methyl ethyl ketone), adding an alcohol, and introducing a Lewis acid to provide the corresponding tetrazolyl compound and an ether (which results from reacting the protective group of the N-protected tetrazolyl compound with the alcohol). The reaction product can optionally be subjected to additional processing steps, including, for example, extraction(s), washing(s), and/or concentration(s). Thereafter, an aliphatic hydrocarbon solvent can be added and the tetrazolyl compound can be crystallized with high efficiency and in good yield. The resulting product can optionally be purified by suspending it in an organic acetate solvent (e.g., isopropyl acetate or ethyl acetate), in a mixture of water and an alcohol (e.g., water and ethanol) and can be recrystallized from a mixture of water and a ketone (e.g., water and acetone).

Scheme 1 illustrates the preparation of a tetrazolyl compound prepared according to one aspect of the invention.

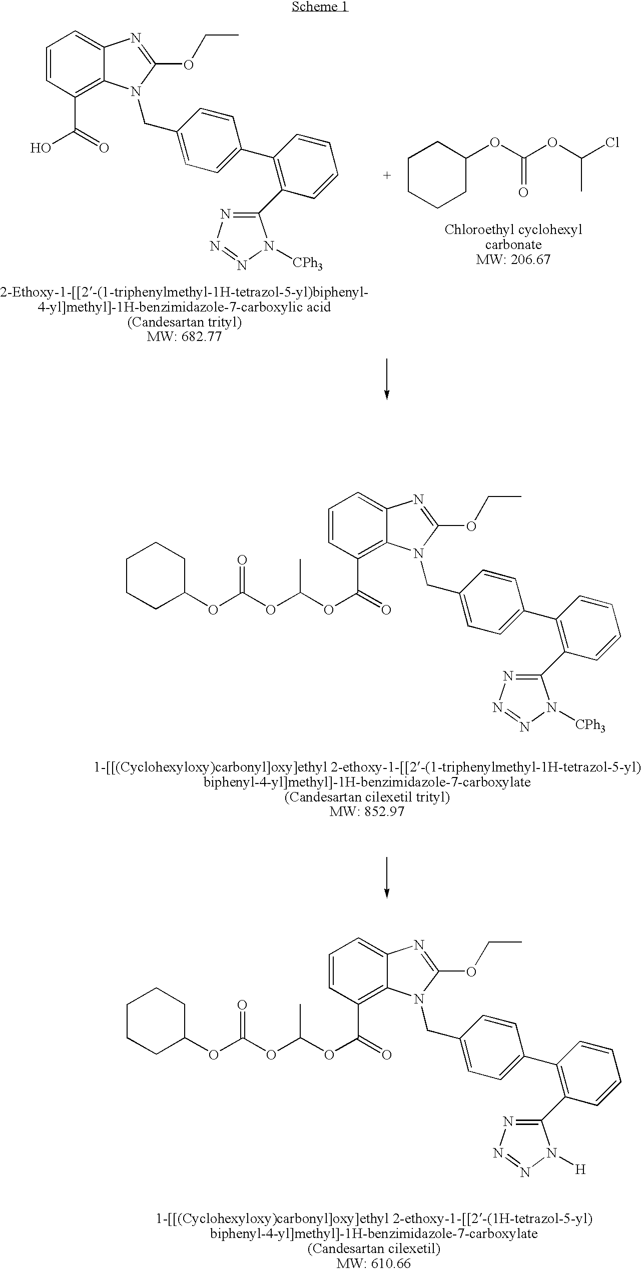

It is believed that the tetrazolyl compound can be efficiently crystallized because the ether in the reaction mixture is highly lipophilic relative to the tetrazolyl compound and is dissolved in the aliphatic hydrocarbon solvent. Additionally, the above-described process is applicable, and still results in good yields, when the N-protected tetrazolyl compound contains other moieties liable to be removed by acid hydrolysis (e.g., an esterified carboxyl group and/or an alkoxy group). Thus, although virtually any N-protected tetrazolyl compound can suitably be used in the invention, the invention is particularly useful when the starting N-protected tetrazolyl compound has at least one other hydrolysable group in addition to the N-protective group of the N-protected tetrazolyl compound.

Suitable inert solvents for use in the invention include any solvent that does not take part in the reaction and is capable of dissolving the N-deprotected tetrazolyl compound. Preferred inert solvents include, for example, aromatic solvents (e.g., toluene), ethers (e.g., tetrahydrofuran) and ketones (e.g., acetone or methyl ethyl ketone). Methyl ethyl ketone is a particularly preferred inert solvent. The total amount of the inert solvent used is not critical, but must be at least an amount sufficient to dissolve the N-deprotected tetrazolyl compound.

Suitable Lewis acids for use in the invention include, for example, AlCl₃, TiCl₄, ZnBr₂ and, preferably, ZnCl₂. The total amount of the Lewis acid used is not critical, but is approximately 1 to approximately 3 equivalents, and more preferably approximately 1.5 equivalents, per mole of the N-protected tetrazolyl compound.

Suitable alcohols for use in the invention include lower alcohols (e.g., C₁-C₄) including, for example, ethanol and, preferably, methanol. The total amount of alcohol used is not critical, but is approximately 1 mole, preferably approximately 2 to approximately 100 moles, and more preferably approximately 5 to approximately 50 moles, per mole of the N-protected tetrazolyl compound.

In the invention, the N-protected tetrazolyl compound is generally first combined and dissolved in the inert solvent. Next, the Lewis acid is added, followed by addition of the alcohol. These steps, however, can be carried in different sequences (e.g., (i) N-protected tetrazolyl, (ii) ZnCl₂, (iii) inert solvent and (iv) alcohol). The reaction temperature is not critical but generally ranges from between approximately room temperature to the boiling point of the solvent, and more preferably at approximately 40° C. The reaction time, which is not particularly restricted, is generally between approximately 1 to 24 hours, and more preferably approximately 2 hours.

The reaction product(s) can optionally be subjected to additional processing steps, including, for example, extraction(s), washing(s), concentration(s), filtrations(s) and/or similar steps. For example, the solution can be filtered to remove insolubles or treated with a decolorizing agent (e.g., activated charcoal, alumina, silica gel) in order to improve its color.

The resulting tetrazolyl compound can be crystallized from an aliphatic hydrocarbon solvent. Suitable aliphatic hydrocarbon solvents include, for example, pentane, hexane, heptane and others. Hexane is a more preferred solvent, and heptane is a particularly preferred solvent.

The resulting product can optionally be purified by suspending it in an organic acetate solvent including and preferably in, for example, isopropyl acetate or ethyl acetate and/or in an alcohol solvent including, for example, methanol or ethanol and/or in a mixture of water and an alcohol and preferably in a water/ethanol mixture. The resulting product can also optionally be recrystallized from a mixture of water and a ketone, preferably a water/acetone mixture.

The resulting crystals of the deprotected tetrazolyl compounds are of high quality and are obtained in good yields.

Compounds that can be produced in accordance with the process of the invention include, for example, losartan, irbesartan, valsartan and, preferably, candesartan cilexetil.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### Example/STEP 1

### Preparation of 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., Candesartan cilexetil trityl)

### Example/Step 1-A

To a 1 L, three-necked round-bottomed spherical flask equipped with a reflux condenser and a thermometer, were added 100.0 g of 2-ethoxy-1-[[2′-(1-triphenylmethyl-1H-tetrazol-5-yl)biphenyl-4-yl]methyl]-1H-benzimidazole-7-carboxylic acid (146.5 mmol), chloroethyl cyclohexyl carbonate (approximately 36.32 g, 175.8 mmol), potassium carbonate (approximately 24.29 g, 175.8 mmol), benzyltriethylammonium chloride (approximately 8.340 g, 36.62 mmol) and tetrahydrofuran (approximately 350 mL). The suspension is heated to reflux (approximately 63-65° C.) over approximately 30 minutes and maintained at this temperature for approximately 5 hours. The heating was stopped, and the suspension was cooled to approximately 20-25° C. over approximately 30 minutes. The suspension was filtered, and the resulting white solid was washed with tetrahydrofuran (2× approximately 100 mL). The solid was discarded, and the yellow mother liquors were concentrated by distilling off the tetrahydrofuran under vacuum. Isopropyl alcohol (approximately 500 mL) was added to the concentrated solution, and the suspension was heated to reflux (approximately 76-78° C.). After reaching reflux temperature, the solution was allowed to cool to approximately 20-25° C. over approximately 3 hours during which time a solid precipitates. The resulting suspension was then stirred at approximately 20-25° C. for approximately 1 hour. The suspension was then filtered, and the resulting solid was washed with isopropyl alcohol (2× approximately 50 mL) to yield 171.0 g (loss on drying (“LOD”)=23.21%, 131.3 g (dry), quantitative yield) of crude 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl). Table 1 illustrates the results of an HPLC analysis of the crude candesartan cilexetil trityl obtained in Example/Step 1-A.

TABLE 1

| | | 2-ethoxy-1-[[2′-(1-triphenylmethyl- | |
|---|---|---|---|
| | | 1H-tetrazol-5-yl)biphenyl- | Unknown |
| | Candesartan | 4-yl]methyl]-1H-benzimidazole- | Impurity |
| | Cilexetil | 7-carboxylic acid | (Run Time = |
| HPLC | Trityl | (Candesartan trityl) | ~30 minutes) |
| % Area | 97.809 | 0.127 | 0.059 |

### Example/Step 1-B

To a 1 L, three-necked round-bottomed spherical flask, equipped with a reflux condenser and a thermometer, are added 171.0 grams of the candesartan cilexetil trityl obtained in example/step 1-A and isopropyl alcohol (approximately 625 mL). The suspension was heated to reflux (approximately 80-82° C.) and stirred at this temperature for approximately 10 minutes. The suspension was then cooled to approximately 20-25° C. over approximately 2 hours. After stirring at approximately 20-25° C. for approximately 1 hour, the suspension was filtered, and the resulting solid was washed with isopropyl alcohol (2× approximately 125 mL). The resulting solid was dried under vacuum at approximately 40° C. until constant weight to yield 123.3 g of candesartan cilexetil trityl. Table 2 illustrates the results of an HPLC analysis of candesartan cilexetil trityl obtained in Example/Step 1-B.

TABLE 2

| | | 2-ethoxy-1-[[2′-(1-triphenylmethyl- | Unknown |
|---|---|---|---|
| | Candesartan | 1H-tetrazol-5-yl)biphenyl- | Impurity |
| | Cilexetil | 4-yl]methyl]-1H-benzimidazole-7- | (Run Time = |
| HPLC | Trityl | carboxylic acid (Candesartan trityl) | ~30 minutes) |
| % Area | 98.641 | 0.032 | 0.010 |

### Example/STEP 2

### Preparation of 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., Candesartan cilexetil)

### Example/Step 2-A

To a 250 mL, three-necked round-bottomed spherical flask, equipped with a reflux condenser and a thermometer, are added 10.00 g of candesartan cilexetil trityl (11.72 mmol) prepared as described in example/step 1, zinc chloride (3.196 g, 23.45 mmol), methylethylketone (54.0 mL) and methanol (6.0 mL). The candesartan cilexetil trityl used in this example/step can either be dry (as described in example/step 1) or can, alternatively, be used wet of isopropyl alcohol. The suspension was stirred at approximately 23-25° C. for approximately 4 hours. The suspension was then heated to approximately 40° C. (±2° C.) over approximately 30 minutes and maintained at this temperature for approximately 1 hour. The heating was stopped, and the resulting solution was cooled to approximately 20-25° C. over about 20 minutes. Deionized water (approximately 54 mL) was then added to the yellow solution. The aqueous layer was separated, and to the organic layer was added n-heptane (approximately 50 mL). The solution was seeded with candesartan cilexetil (polymorph I) and was stirred overnight at room temperature. The suspension was then cooled to approximately 10-12° C. and stirred at this temperature for approximately 1 hour. The suspension was then filtered, and the resulting white solid was washed with n-heptane (2× approximately 10 mL) to yield 7.14 g (LOD=19.99%, 5.71 g (dry), 79.75% yield) of crude 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., candesartan cilexetil). Table 3 illustrates the results of an HPLC analysis of the crude candesartan cilexetil obtained in Example/Step 2-A.

TABLE 3

| | | | 1-[[(Cyclohexyloxy) | |
|---|---|---|---|---|
| | | | carbonyl]oxy]ethyl | |
| | | | 2-hydroxy-1-[[2′-(1H- | |
| | | | tetrazol-5-yl)biphenyl- | |
| | | | 4-yl]methyl]-1H- | |
| | | | benzimidazole- | Methyl |
| | Candesartan | Candesartan | 7-carboxylate | Trityl |
| HPLC | Cilexetil | Cilexetil Trityl | (Desethyl impurity) | Ether |
| % Area | 95.185 | 0.353 | 3.049 | 0.425 |

### Example/Step 2-B

To a 100 mL, three-necked round-bottomed spherical flask, equipped with a thermometer, are added 6.25 g of the candesartan cilexetil (8.19 mmol) obtained in example/step 2-A and isopropyl acetate (approximately 40.0 mL). The suspension was stirred at approximately 20-22° C. for approximately 2 hours and then further cooled and stirred at approximately 10-12° C. for approximately 1 hour. The suspension was then filtered, and the resulting white solid was washed with isopropyl acetate (2× approximately 5 mL) to yield 4.47 g of candesartan cilexetil (LOD=16.27%, 3.70 g (dry), 74.00% yield). Table 4 illustrates the results of an HPLC analysis of the candesartan cilexetil obtained in Example/Step 2-B.

TABLE 4

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 98.934 | 0.069 | 0.414 | 0.032 |

### Example/Step 2-C

In a 100 mL, three-necked round-bottomed spherical flask, equipped with a thermometer, are added 3.58 g of the candesartan cilexetil (4.91 mmol) obtained in example/step 2-B and ethanol (approximately 15.0 mL). The suspension was heated to approximately 40° C., and water (approximately 7.5 mL) was added. The suspension was stirred at approximately 40° C. for approximately 30 minutes. Thereafter, the suspension was cooled to approximately 10-12° C. and stirred at this temperature for approximately 1 hour. The suspension was then filtered, and the resulting white solid was washed with water (2× approximately 5 mL). The resulting solid was dried under vacuum at approximately 30° C. until constant weight to yield 2.13 g (71.00% yield) of candesartan cilexetil. Table 5 illustrates the results of an HPLC analysis of the candesartan cilexetil obtained in Example/Step 2-C.

TABLE 5

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 99.129 | N.D. | 0.267 | N.D. |

### Example/STEP 3

### Preparation of 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., Candesartan cilexetil)

### Example/Step 3-A

To a 2 L, three-necked round-bottomed spherical flask, equipped with a reflux condenser and a thermometer, are added 123.3 g of candesartan cilexetil trityl (144.5 mmol) prepared as described in example/step 1, zinc chloride (approximately 23.64 g, 173.4 mmol), methylethylketone (approximately 665 mL) and methanol (approximately 74 mL). The suspension was heated to approximately 40° C. (±2° C.) over approximately 15 minutes and maintained at this temperature for approximately 2 hours. The heating was stopped, and the resulting solution was cooled to approximately 20-25° C. over approximately 15 minutes. Deionized water (approximately 665 mL) and ammonium chloride (approximately 59.6 g) were then added to the yellow solution. The aqueous layer was then separated, and to the organic layer was added n-heptane (approximately 924 mL). The mixture was stirred at room temperature for approximately 24 hours. The suspension was then cooled to approximately 0-5° C. and was stirred at this temperature for approximately 2 hours. The suspension was then filtered, and the resulting solid was washed with n-heptane (2× approximately 103 mL) to yield 103.5 g (LOD=18.81%; 84.03 g (dry) 95.23% yield) of crude candesartan cilexetil. Table 6 illustrates the results of an HPLC analysis of the crude candesartan cilexetil obtained in Example/Step 3-A.

TABLE 6

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 93.982 | 0.243 | 2.124 | 0.317 |

### Example/Step 3-B

To a 1 L, three-necked round-bottomed spherical flask, equipped with a reflux condenser and a thermometer, are added 103.5 g of candesartan cilexetil (137.6 mmol) obtained in example/step 3-A and ethanol (approximately 421 mL). The suspension was heated to approximately 40° C. (±2° C.), and deionized water (approximately 291 mL) was added. The suspension was stirred at approximately 40° C. for approximately 30 minutes. Thereafter, the suspension was cooled to approximately 5-10° C. and maintained at this temperature for approximately 2 hours. The suspension was filtered, and the resulting solid was washed with water (2× approximately 22 mL) to yield 110.2 g (LOD=30.00%; 77.17 g (dry), 91.81% yield) of candesartan cilexetil. Table 7 illustrates the results of an HPLC analysis of the candesartan cilexetil obtained in Example/Step 3-B.

TABLE 7

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 97.463 | 0.238 | 0.908 | 0.115 |

### Example/Step 3-C

To a 1 L, three-necked round-bottomed spherical flask, equipped with a reflux condenser and a thermometer, are added 110.2 g of candesartan cilexetil (126.4 mmol) obtained in example/step 3-B and acetone (approximately 338 mL). The suspension was stirred at reflux, and, after reaching reflux, water (approximately 180 mL) was added. Thereafter, the mixture was stirred at reflux for approximately 30 minutes. The suspension was then cooled to approximately 0-5° C. and stirred at this temperature for approximately 3 hours. The suspension was then filtered, and the resulting white solid was washed with water (2× approximately 34 mL) to yield 93.21 g (LOD=18.63%; 75.85 g (dry), 98.31% yield) of candesartan cilexetil. Table 8 illustrates the results of an HPLC analysis of the candesartan cilexetil obtained in Example/Step 3-C.

TABLE 8

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 99.082 | 0.342 | 0.174 | 0.014 |

### Example/Step 3-D

In a 500 mL, three-necked round-bottomed spherical flask, equipped with a thermometer, are added 93.21 g of the candesartan cilexetil (124.3 mmol) obtained in example/step 3-C and acetone (approximately 296 mL). The suspension was stirred at reflux, and water (approximately 152 mL) was added. The mixture was stirred at reflux for approximately 30 minutes. The suspension was then cooled to approximately 0-5° C. and stirred at this temperature for approximately 3 hours. The suspension was then filtered, and the resulting white solid was washed with water (2× approximately 35 mL). The resulting solid was dried under vacuum at approximately 30° C. until constant weight to yield 71.17 g (93.79% yield) of candesartan cilexetil. Table 9 illustrates the results of an HPLC analysis of the prepared candesartan cilexetil obtained in Example/Step 3-D.

TABLE 9

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl impurity) | Ether |
| % Area | 99.461 | 0.043 | 0.130 | N.D. |

### Example/STEP 4

### Preparation of 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., Candesartan cilexetil)

### Example/Step 4-A

To a 100 mL, three-necked round-bottomed spherical flask, equipped with a reflux condenser and a thermometer, are added 5.00 g of candesartan cilexetil trityl (5.86 mmol) prepared as described in example/step 1, zinc chloride (approximately 0.96 g, 7.03 mmol), methylethyl ketone (approximately 27 mL), methanol (approximately 3.0 mL) and water (approximately 0.2 mL). The suspension was heated to approximately 40° C. (±2° C.) over approximately 15 minutes and maintained at this temperature for approximately 2 hours. The heating was stopped, and the resulting solution was cooled to approximately 20-25° C. over approximately 15 minutes. Deionized water (approximately 27 mL) and ammonium chloride (approximately 2.42 g) were added to the yellow solution. The aqueous layer was separated, and to the organic layer was added n-heptane (approximately 38 mL). The mixture was stirred at room temperature for approximately 20 hours. The suspension was then cooled to approximately 0-5° C. and stirred at this temperature for approximately 2 hours. The suspension was then filtered, and the resulting solid was washed with n-heptane (2× approximately 5 mL). The resulting solid was dried under vacuum at approximately 40° C. until constant weight to yield 2.60 g (72.63% yield) of candesartan cilexetil. Table 10 illustrates the results of an HPLC analysis of the prepared candesartan cilexetil obtained in Example/Step 4-A.

TABLE 10

| | | | 1-[[(Cyclohexyloxy)carbonyl]oxy]ethyl | |
|---|---|---|---|---|
| | | | 2-hydroxy-1-[[2′-(1H-tetrazol-5-yl)biphenyl- | Methyl |
| | Candesartan | Candesartan | 4-yl]methyl]-1H-benzimidazole-7-carboxylate | Trityl |
| HPLC | Cilexetil | Cilexetil Trityl | (Desethyl Impurity) | Ether |
| % Area | 93.861 | 0.172 | 3.195 | N.D. |

### Example/STEP 5

### Preparation of 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., Candesartan cilexetil trityl)

### Example/Step 5-A

To a 50 mL, three-necked round-bottomed spherical flask, equipped with a reflux condenser and a thermometer, was added 5.00 g of 2-ethoxy-1-[[2′-(1-triphenylmethyl-1H-tetrazol-5-yl)biphenyl-4-yl]methyl]-1H-benzimidazole-7-carboxylic acid (7.32 mmol), chloroethyl cyclohexyl carbonate (approximately 1.82 g, 8.79 mmol), potassium carbonate (approximately 1.22 g, 8.79 mmol) and dimethyl sulfoxide (approximately 13 mL). The suspension was heated to 60° C. (±2° C.) over approximately 1 hour and maintained at this temperature for approximately 3 hours. The heating was stopped, and the suspension was cooled to approximately 20-25° C. over approximately 1 hour. The suspension was then filtered, and the resulting white solid was washed with dimethyl sulfoxide (3× approximately 10 mL). The resulting solid was dried under vacuum at approximately 40° C. until constant weight to yield 8.42 g (>100% yield) of crude candesartan cilexetil trityl. Table 11 illustrates the results of an HPLC analysis of the crude candesartan cilexetil trityl obtained in Example/Step 5-A.

TABLE 11

| | | 2-ethoxy-1-[[2′-(1- | |
|---|---|---|---|
| | | triphenylmethyl-1H- | |
| | | tetrazol-5-yl)biphenyl- | |
| | | 4-yl]methyl]-1H- | Unknown |
| | Candesartan | benzimidazole-7- | Impurity |
| | Cilexetil | carboxylic acid | (Run Time = |
| HPLC | Trityl | (Candesartan trityl) | ~30 minutes) |
| % Area | 96.579 | 0.022 | 0.091 |

### Example/STEP 6

### Preparation of 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., Candesartan cilexetil trityl)

### Example/Step 6-A

To an appropriate vessel was added 15.0 Kg of 2-ethoxy-1-[[2′-(1-triphenylmethyl-1H-tetrazol-5-yl)biphenyl-4-yl]methyl]-1H-benzimidazole-7-carboxylic acid (22.0 mol), chloroethyl cyclohexyl carbonate (approximately 5.46 Kg, 26.4 mol), potassium carbonate (approximately 3.64 Kg, 26.4 mol) and N-methyl-2-pyrrolidinone (approximately 40 Kg). The suspension was heated to 60° C. (±3° C.) over approximately 30 minutes and maintained at this temperature for approximately 3 hours. The heating was stopped, and the suspension was cooled to approximately 20-25° C. over approximately 30 minutes. The suspension was then filtered, and the resulting white solid was washed with N-methyl-2-pyrrolidinone (approximately 16 Kg) and methylethylketone (3× approximately 46 Kg). The solid was discarded, and the yellow mother liquors were concentrated by removing the methylethylketone by distillation under vacuum. Next, methanol (approximately 117 Kg) was added to the concentrated solution. The mixture was then cooled to 0-5° C. and stirred at this temperature for 2 hours, during which time a solid precipitates. The suspension was then filtered and the resulting solid was washed with methanol (approximately 10 Kg) to yield 20.65 Kg (LOD=21.14%, 16.28 Kg (dry), 86.87% yield) of crude 1-[[(cyclohexyl oxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl). Table 12 illustrates the results of an HPLC analysis of the crude candesartan cilexetil trityl obtained in Example/Step 6-A.

TABLE 12

| | | 2-ethoxy-1-[[2′-(1- | |
|---|---|---|---|
| | | triphenylmethyl-1H- | |
| | | tetrazol-5- | |
| | | yl)biphenyl-4- | |
| | | yl]methyl]-1H- | Unknown |
| | Candesartan | benzimidazole-7- | Impurity |
| | Cilexetil | carboxylic acid | (Run Time = |
| HPLC | Trityl | (Candesartan trityl) | ~30 minutes) |
| % Area | 98.109 | N.D. | 0.028 |

### Example/Step 6-B

To an appropriate vessel was added 20.65 Kg of the candesartan cilexetil trityl obtained in example/step 6-A and isopropyl acetate (approximately 118 Kg). The suspension was heated to reflux temperature (approximately 85-87° C.) and stirred at this temperature for approximately 10 minutes. The suspension was then cooled to approximately 0-5° C. over approximately 2 hours. After stirring at approximately 0-5° C. for approximately 2 hours, the suspension was filtered and the resulting solid was washed with isopropyl acetate (approximately 7.5 Kg) to yield 13.15 Kg (LOD=10.12%, 11.82 Kg (dry), 72.60% yield) of crude 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl). Table 13 illustrates the results of an HPLC analysis of the crude candesartan cilexetil trityl obtained in Example/Step 6-B.

TABLE 13

| | | 2-ethoxy-1-[[2′-(1- | |
|---|---|---|---|
| | | triphenylmethyl-1H- | |
| | | tetrazol-5- | |
| | | yl)biphenyl-4- | |
| | | yl]methyl]-1H- | Unknown |
| | Candesartan | benzimidazole-7- | Impurity |
| | Cilexetil | carboxylic acid | (Run Time = |
| HPLC | Trityl | (Candesartan trityl) | ~30 minutes) |
| % Area | 98.63 | N.D. | n.d. |

### Example/STEP 7

### Preparation of 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., Candesartan cilexetil)

### Example/Step 7

To an appropriate vessel were added approximately 44.00 Kg of candesartan cilexetil trityl (obtained following the procedure described in Example/Step 6), zinc chloride (approximately 8.48 Kg), methylethylketone (approximately 192 Kg) and methanol (approximately 21 Kg). The mixture was heated to 40-45° C., and stirred at this temperature for approximately 2 hours. The resulting solution was then cooled to approximately 20-25° C. and was washed twice with an aqueous solution of ammonium chloride. n-Heptane (approximately 218 Kg) was added to the organic layer, and the mixture was stirred at 0-5° C. for approximately 24 hours. The suspension was then filtered, and the resulting solid was washed with n-heptane (approximately 20 Kg).

The resulting solid was next suspended in ethanol (approximately 121 Kg) and was heated to 40° C. Water (approximately 104 Kg) was added to the suspension, and it was stirred at 40° C. for 30 minutes. The suspension was then cooled to 5-10° C. and stirred at this temperature for 2 hours. The suspension was filtered, and the solid was washed with water (approximately 8 Kg). The resulting solid was next suspended in acetone (approximately 88 Kg) and was heated to reflux to form a solution. Water (approximately 55 Kg) was then added to the solution, and the final suspension was stirred at reflux for 10 minutes. The suspension was then cooled to 0-5° C., and it was stirred at this temperature for 3 hours. The suspension was filtered and the solid was washed with water (approximately 8 Kg). The solid was then suspended in acetone (approximately 87 Kg) and heated to reflux. The resulting solution was filtered and again heated to reflux. Water (approximately 54 Kg) was added to the solution and it was stirred at reflux for 10 minutes. The suspension was then cooled to 0-5° C., and it was stirred at this temperature for 3 hours. The suspension was filtered and the solid was washed with water (approximately 8 Kg). The solid was suspended in methanol (approximately 40 Kg) and it was stirred at 20-25° C. for 30 minutes. The suspension was filtered and the solid was washed with methanol (approximately 4 Kg). The solid was then suspended in ethyl acetate (approximately 33 Kg), and it was heated to 40° C. The suspension was stirred at 40° C. for 30 minutes, cooled to 0-5° C. and stirred at this temperature for 1 hour. After filtering the suspension, the resulting solid was washed with ethyl acetate (approximately 4 Kg) and dried at 40° C. under vacuum to yield 16.35 Kg of 1-[[(cyclohexyloxy)carbonyl]oxy]ethyl 2-ethoxy-1-[[2′-(1H-tetrazol-5-yl)biphenyl-4-yl]methyl]-1H-benzimidazole-7-carboxylate (i.e., candesartan cilexetil) (51.60% yield).

Table 14 illustrates the results of particle size determination of candesartan cilexetil obtained in Example/Step 7. Table 15 illustrates the results of particle size determination of candesartan cilexetil obtained in Example/Step 7 after milling.

TABLE 14

| | D₁₀(v) | D₅₀(v) | D₉₀(v) |
|---|---|---|---|
| Particle size | 2.2 microns | 8.0 microns | 20.7 microns |

TABLE 15

| | D₁₀(v) | D₅₀(v) | D₉₀(v) |
|---|---|---|---|
| Particle size | 2.0 microns | 7.1 microns | 19.1 microns |

Table 16 illustrates the results of an HPLC analysis of candesartan cilexetil obtained in Example/Step 7. Table 17 illustrates the result of specific surface area determination of candesartan cilexetil obtained in Example/Step 7 and Table 18 illustrates more analytical data of candesartan cilexetil obtained in Example/Step 7.

TABLE 16

| | | | 1-[[(Cyclohexyloxy) carbonyl]oxy]ethyl | | |
|---|---|---|---|---|---|
| | | | 2-hydroxy-1-[[2′-(1H-tetrazol-5-yl)biphenyl- | | Other |
| | Candesartan | Candesartan | 4-yl]methyl]-1H-benzimidazole-7-carboxylate | Methyl | Unknown |
| HPLC | Cilexetil | Cilexetil Trityl | (Desethyl impurity) | Trityl Ether | Impurities |
| % Area | 99.67 | 0.064 | 0.058 | ND | <0.1% |

TABLE 17

| | |
|---|---|
| Specific Surface Area | 2.9940 ± 0.0234 m²/g |

TABLE 18
Analytical Data Candesartan Cilexetil

| | |
|---|---|
| Assay (HClO₄) | 99.51% |
| Water Content (K.F.) | 0.07% |
| Loss on Drying | 0.02% |
| Sulphated Ashes | <0.1% |
| Heavy Metals | <10 ppm |
| Residual Solvents | Methanol < 100 ppm |
| | Ethanol = 95 ppm |
| | Acetone = 154 ppm |
| | Ethyl Acetate = 389 ppm |

By following the procedure as disclosed in Example/Step 7 more examples of candesartan cilexetil were prepared. Table 19 illustrates the results of specific surface area determination of candesartan cilexetil obtained in these examples.

TABLE 19

| Example | Specific Surface Area |
|---|---|
| Example/Step 8 | 1.3336 ± 0.0241 m²/g |
| Example/Step 9 | 1.7939 ± 0.0211 m²/g |
| Example/Step 10 | 1.5759 ± 0.0227 m²/g |

In each of the foregoing examples/steps, the chromatographic separation (i.e., HPLC analysis) was performed using a Waters Symmetry C18, 3.5 μm, 10 cm×4.6 mm. I.D column. The mobile phase A was 0.010 M ammonium formate buffer (NH₄COOH), pH=4.0, which was prepared by dissolving 0.63 g of NH₄COOH in 1000 mL of water, adjusting the pH to 4.0 with formic acid and then filtering through 0.22 μm nylon filter under vacuum. The mobile phase B was acetonitrile. The chromatograph was equipped with a 225 nm detector.

The chromatograph was programmed as follows: 0-3 minutes isocratic 60% mobile phase A and 40% mobile phase B; 3-15 minutes linear gradient to 10% mobile phase A; 15-40 minutes isocratic 10% mobile phase A; 40-45 minutes linear gradient to 60% mobile phase A; and 45-55 minutes equilibration with 60% mobile phase A. The flow rate was 1.0 mL per minute at room temperature, and test samples (10 μL) were prepared by dissolving the appropriate amount of sample to obtain a concentration of 1 mg of sample per mL of acetonitrile.

Particle size was measured using a Malvern Mastersizer S particle size analyzer with an MS1 Small Volume Sample Dispersion unit attached using a 300RF mm lens and a beam length of 2.4 mm. Samples for analysis were prepared by dispersing a weighed amount of candesartan cilexetil (approximately 25 mg) in 20 mL of Isopar G. The samples were sonicated for 15 seconds and delivered drop-wise to a background corrected measuring cell previously filled with Isopar G until the obscuration reached the desired level (11-12%). The dispersion placed into the measuring cell was sonicated for 1 minute. Volume distributions were obtained for three times. Upon measurement completion, the sample cell was emptied, cleaned and refilled with suspending medium and the sampling procedure was then repeated. For characterization, the values of D₁₀, D₅₀ and D₉₀ were specifically listed, each one being the mean of the six values available for each characterization parameter.

The BET (Brunauer, Emmett and Teller) specific surface for candesartan cilexetil was measured using Micromeritics ASAP2010 equipment. Samples for analysis were degassed at 110° C. under vacuum for two hours. The determination of N₂ adsorption at 77° K of weighted samples (400 mg of candesartan cilexetil (approximately 0.4 g)) was measured for relative pressures in the range of 0.07-0.2.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A process for preparing candesartan cilexetil and related tetrazolyl compounds comprising:
(i) removing a protective group from an N-protected tetrazolyl compound by solvolysis using a Lewis acid in an inert solvent and in the presence of an alcohol; and
(ii) isolating said candesartan cilexetil and related tetrazolyl compounds.

2. The process of claim 1, wherein said protecting group is a triphenylmethane (trityl) protecting group.

3. The process of claim 1, wherein said Lewis Acid is at least one of AlCl₃, TiCl₄, ZnBr₂, ZnCl₂ and combinations thereof.

4. The process of claim 1, wherein said Lewis acid is ZnCl₂.

5. The process of claim 1, wherein the amount of said is Lewis Acid is approximately 1 to approximately 3 equivalents per mole of said N-protected tetrazolyl compound.

6. The process of claim 1, wherein the amount of said is Lewis Acid is approximately 1.5 equivalents per mole of said N-protected tetrazolyl compound.

7. The process of claim 1, wherein said inert solvent is at least one of toluene, tetrahydrofuran, acetone, methyl ethyl ketone and mixtures thereof.

8. The process of claim 1, wherein said alcohol is a lower alcohol having between 1 and 4 carbons.

9. The process of claim 1, wherein the amount of said alcohol is approximately 1 mole per mole of said N-protected tetrazolyl compound.

10. The process of claim 1, wherein the amount of said alcohol is approximately 2 to approximately 100 moles per mole of said N-protected tetrazolyl compound.

11. The process of claim 1, wherein the amount of said alcohol is approximately 5 to approximately 50 moles per mole of said N-protected tetrazolyl compound.

12. The process of claim 1, further comprising at least one additional processing step.

13. The process of claim 12, wherein said at least one additional process step comprises at least one of an extraction step, a washing step, a concentration step, a crystallization step and a recrystallization step.

14. The process of claim 13, where said recrystallization step comprises recrystallizing from a mixture of water and a ketone.

15. The process of claim 14, wherein said ketone is acetone.

16. The process of claim 13, further comprising the step of purifying said isolated candesartan cilexetil and related tetrazolyl compounds by suspending said isolated candesartan cilexetil and related tetrazolyl compounds in at least one of an organic acetate solvent, an alcohol, a mixture of water and said alcohol and mixtures thereof.

17. The process of claim 16 wherein said organic acetate solvent is at least one of isopropyl acetate, ethyl acetate and mixtures thereof and wherein said alcohol is at least one of methanol, ethanol and mixtures thereof.

18. The process of claim 1, further comprising preparing 1-[[(cyclohexyloxy)carbonyl]oxy]ethyl 2-ethoxy-1-[[2′-(1-triphenylmethyl-1H-tetrazol-5-yl)biphenyl-4-yl]methyl]-1H-benzimidazole-7-carboxylate (i.e., candesartan cilexetil trityl) as an intermediate by condensing 2-ethoxy-1-[[2′-(1-triphenylmethyl-1H-tetrazol-5-yl)biphenyl-4-yl]methyl]-1H-benzimidazole-7-carboxylic acid with chloroethyl cyclohexyl carbonate in at least one high-boiling organic solvent in the presence of potassium carbonate.

19. The process of claim 18, wherein said at least one high-boiling organic solvent comprises at least one of N-methyl-2-pyrrolidinone (NMP), dimethyl sulfoxide (DMSO) and mixtures thereof.

20. The use of candesartan cilexetil and related tetrazolyl compounds made according to the process of claim 1 to treat hypertension.

21. The use of candesartan cilexetil and related tetrazolyl compounds made according to the process of claim 1 to treat at least one circulatory disease.

22. The use of claim 21, wherein said at least one circulatory disease is at least one of heart failure, stroke, cerebral apoplexy, nephropathy and nephritis.

23. A formulation comprising candesartan cilexetil and related tetrazolyl compounds made according to the process of claim 1.

24. The formulation of claim 23, wherein said candesartan cilexetil and related tetrazolyl compounds have an approximate particle size of D₉₀≦approximately 25 μm.

25. The formulation of claim 23, wherein said candesartan cilexetil and related tetrazolyl compounds have an approximate particle size of D₅₀≦approximately 10 μm.

26. The formulation of claim 23, wherein said candesartan cilexetil and related tetrazolyl compounds have an approximate particle size of D₁₀≦approximately 3 μm.

27. Candesartan cilexetil and related tetrazolyl compounds having a specific surface area of approximately 1 to approximately 3 m²/g.

28. A formulation comprising said candesartan cilexetil and related tetrazolyl compounds according to claim 27.

29. Candesartan cilexetil and related tetrazolyl compounds having less than approximately 0.1% by weight of residual solvent.

30. A formulation comprising said candesartan cilexetil and related tetrazolyl compounds according to claim 29.
